(19) United States
(12) Patent Application Publication
(10) Pub. No.: US 2004/0170683 A1
(43) Pub. Date: Sep. 2, 2004

(54) Tablets comprising modafinil

(76) Inventor: Bernard Charles Sherman, Toronto (CA)

Correspondence Address: NIXON & VANDERHYE, PC, 1100 N GLEBE ROAD, 8TH FLOOR, ARLINGTON, VA, 22201-4714, US

(21) Appl. No.: 10/781,815

(22) Filed: Feb. 20, 2004

(30) Foreign Application Priority Data
Feb. 28, 2003 (CA) 2,420,180

(51) Int. Cl. A61K 9/20; A61K 31/165

(52) U.S. Cl. 424/465; 514/618

(57) ABSTRACT

A pharmaceutical tablet comprising modafinil particles, wherein more than 5% of the modafinil particles have a diameter of more than 200 microns, less than 5% of the modafinil particles have a diameter of more than 800 microns, and the tablet has a disintegration time of less than 20 minutes.

### BACKGROUND OF THE INVENTION

Modafinil is a drug disclosed U.S. Pat. No. 4,177,290 used for treatment of idiopathic hypersomnia and narcolepsy. It is sold in the United States under the tradename Provigil™ in tablets comprising 100 mg and 200 mg modafinil per tablet.

U.S. Pat. No. RE37,516E discloses that the extent of absorption of modafinil upon oral administration depends on the particle size distribution of the modafinil in the composition. It is disclosed that, in the first human trials of modafinil to treat narcolepsy, the modafinil had a median particle size between 80 and 150 microns. Subsequent trials with modafinil having a median particle size between 30 and 50 microns gave greater extent of absorption. FIG. 7 in this patent confirms that the dissolution of capsules containing the finer particles is substantially faster than for capsules containing the coarser particles.

This patent specifically claims any composition wherein at least 95% of the cumulative total of modafinil particles have a diameter of less than about 200 microns.

One problem with compositions claimed in U.S. Pat. No. RE37,516E is that, when modafinil is milled to have at least 95% of the particles below 200 microns, it is too fine to be free-flowing, and this complicates the process of making tablets comprising such modafinil.

An objective of the present invention is thus to enable modafinil tablets comprising coarser modafinil, which still exhibit relatively rapid dissolution rate.

### BRIEF SUMMARY OF THE INVENTION

It has been found that tablets comprising modafinil particles such that more than 5% of the modafinil particles have a diameter of more than 200 microns can still have a rapid dissolution rate, comparable to that of Provigil™ tablets, provided that less than 5% of the modafinil particles have a diameter of more than 800 microns and the tablets has a disintegration time of less than 20 minutes.

### DETAILED DESCRIPTION OF THE INVENTION

For the purpose of the present disclosure and claims, “diameter” of a particle will be understood to mean the diameter of a sphere having the same volume as the particular particle.

Also, “disintegration time” of a tablet will be understood to mean the time for complete disintegration in water as determined using the apparatus and procedure set out in the U.S. Pharmacopoeia and National Formulary (USP26/NF21).

Compositions of the present invention will be tablets comprising modafinil particles wherein more than 5% of the modafinil particles have a diameter of more than 200 microns, provided that less than 5% of the particles have a diameter of more than 800 microns, and the tablets have a disintegration time of less than 20 minutes.

Preferably less than 5% of the modafinil particles will have a diameter of more than 600 microns. More preferably less than 5% of the modafinil particles will have a diameter of more than 450 microns. Also preferably, more than 5% of the modafinil particles will have a diameter of more than 240 microns.

Also, preferably the tablets will have a disintegration time of less than 10 minutes, more preferably the tablets will have a disintegration time of less than 5 minutes, and most preferably the tablets will have a disintegration time of less than 2 minutes.

Modafinil having essentially all of the particles with a given range can be prepared, beginning with coarse modafinil particles, by the steps of first grinding all of the particles through a first screen with hole size equivalent to the largest desired particle size, and then sifting the resulting particles over a second, finer screen, with hole size equivalent to the smallest desired size, and discarding the particles that go through the second finer screen.

For example, grinding through a #30 screen (30 wires per inch) will result in a maximum particle diameter of about 600 microns, and sifting on a #60 screen (60 wires per inch) will remove particles of diameter below about 240 microns. Hence, by grinding through a #30 screen and then sifting on a #60 screen to remove the fine particles, modafinil can be achieved having essentially all particles smaller than about 600 microns and larger than about 240 microns.

The modafinil with desired particle size range so achieved can then be made into tablets by conventional means. That is to say, the modafinil can be mixed together with a filler and binder such as, for example, microcrystalline cellulose, a disintegrant such as, for example, croscarmellose sodium and a lubricant, such as for example, magnesium stearate. The mixture will then be compressed into tablets on a tablet press.

The invention will be better understood from the following examples, which are intended to be illustrative and not limiting.

### EXAMPLE 1

Modafinil consisting of primarily coarse particles was ground through a #30 screen, and the resulting granules were sifted over a #60 screen. The particles that went through the #60 screen were discarded. The modafinil that remained on the #60 screen was thus modafinil having essentially all particles with diameter between about 240 microns and about 600 microns.

### EXAMPLE 2

Modafinil particles resulting from example 1 were mixed with a binder, disintegrant and lubricant and compressed into tablets comprising 100 mg modafinil per tablet. The amount of disintegrant used was sufficient to cause the disintegration time of the tablets to be under 2 minutes. The dissolution rate of these tablets was compared to that of Provigil™ 100 mg tablets, in U.S. Pharmacopoeia Apparatus 2, using a stir rate of 75 rpm, in 900 mL of 0.1N HCl. It was found that for both the tablets of this example and Provigil™ tablets, the extent of dissolution exceeded 80% at 20 minutes.

1. A pharmaceutical tablet comprising modafinil particles, wherein more than 5% of the modafinil particles have a diameter of more than 200 microns, less than 5% of the modafinil particles have a diameter of more than 800 microns, and the tablet has a disintegration time of less than 20 minutes.

2. A tablet of claim 1 wherein less than 5% of the modafinil particles have a diameter of more than 600 microns.

3. A tablet of claim 1 wherein less than 5% of the modafinil particles have a diameter of more than 450 microns.

4. A tablet of any of claims 1 to 3, wherein more than 5% of the modafinil particles have a diameter of more than 240 microns.

5. A tablet of any of claims 1 to 3 having a disintegration time of less than 10 minutes.

6. A tablet of claim 4 having a disintegration time of less than 10 minutes.

7. A tablet of any of claims 1 to 3 having a disintegration time of less than 5 minutes.

8. A tablet of claim 4 having a disintegration time of less than 5 minutes.

9. A tablet of any of claims 1 to 3 having a disintegration time of less than 2 minutes.

10. A tablet of claim 4 having a disintegration time of less than 2 minutes.
